(19) United States
(12) Patent Application Publication
(10) Pub. No.: US 2018/0353063 A1
(43) Pub. Date: Dec. 13, 2018

(54) OPTICAL TOMOGRAPHIC IMAGING APPARATUS, CONTROL METHOD THEREFOR, PROGRAM THEREFOR, AND OPTICAL TOMOGRAPHIC IMAGING SYSTEM

(71) Applicant: CANON KABUSHIKI KAISHA, Tokyo (JP)

(72) Inventors: Akihito Uji, Kyoto-shi (JP); Nagahisa Yoshimura, Kyoto-shi (JP); Tomoyuki Makihira, Tokyo (JP); Hirofumi Yoshida, Yokohama-shi (JP)

(21) Appl. No.: 15/535,721

(22) PCT Filed: Jan. 7, 2016
(86) PCT No.: PCT/JP2016/051057

§ 371 Date: Jun. 14, 2017

(30) Foreign Application Priority Data
Jan. 9, 2015 (JP) 2015-003421

(51) Int. Cl. A61B 3/10; A61B 5/00; A61B 3/12; A61B 3/14; G01N 21/47

(52) U.S. Cl. CPC A61B 3/102; A61B 5/0066; A61B 3/12; G01N 2021/1787; A61B 5/0073; G01N 21/4795; A61B 3/14

(57) ABSTRACT

Provided is an optical tomographic imaging apparatus including: a determination unit for determining whether or not an optical member for changing a field angle has been inserted between a scanning unit and an object to be inspected to change a field angle of an acquiring area of a tomographic image; and a switching unit for switching a value of at least one parameter among a control parameter of a control portion for the optical tomographic imaging apparatus, a signal processing parameter of a calculation processing portion, an image processing parameter, and an analysis processing parameter, based on a determination result from the determination unit. Accordingly, a preferred tomographic image of the object is acquired by switching values of various parameters to suitable values even when the optical member for changing a field angle is inserted to change the field angle of the acquiring area of the tomographic image.

### TECHNICAL FIELD

The present invention relates to an optical tomographic imaging apparatus configured to image a tomographic image of an object to be inspected, a control method therefor, a program for executing the control method, and an optical tomographic imaging system.

### BACKGROUND ART

There is developed an optical tomographic imaging apparatus (hereinafter referred to as “OCT apparatus”) configured to image a tomographic image of an object to be inspected through use of optical coherence tomography (hereinafter referred to as “OCT”). In the OCT apparatus, an object is irradiated with a measuring light being a low-coherence light, and a scattered light or a reflected light from the object is caused to interfere with a reference light, to thereby obtain an interference light. Then, a frequency component of a spectrum of the interference light is analyzed, to thereby obtain the tomographic image of the object with high resolution. Such an OCT apparatus is suitably used for a fundus inspection for conducting a medical inspection of an eye to be inspected by obtaining a tomographic image of a fundus of the eye to be inspected.

In regard to an ocular disease, it is important to discover a lesion of the fundus at an early stage, and to start treatment to delay the progress of the lesion extending over a wide area of the fundus at an early stage. In particular, a profound effect is exerted on a visual sense when the lesion reaches a macula, which raises a demand that the lesion be discovered even when the lesion exists at a position sufficiently distant from the macula. In order to meet the demand, the OCT apparatus used for the fundus inspection is expected to have a wider field angle.

In PTL 1, there is disclosed a configuration in which an adapter for imaging an anterior ocular segment is attached to an OCT apparatus for imaging a fundus, and when an imaging field angle is changed, a wide angle lens adapter is attached in place of the adapter for imaging an anterior ocular segment. In addition, in this configuration, it is determined whether or not the adapter for imaging an anterior ocular segment is attached, and a result of the determination is displayed on a monitor.

Further, in PTL 2, there is described a configuration in which an adapter for imaging an anterior ocular segment is attached to an OCT apparatus for imaging a fundus. In this configuration, in response to the detection of the attachment of the adapter, a switch is also made from a monitor display screen for imaging a fundus to a monitor display screen for imaging an anterior ocular segment.

### CITATION LIST

### Patent Literature

PTL 1: Japanese Patent Application Laid-Open No. 2011-147609

PTL 2: Japanese Patent Application Laid-Open No. 2013-212313

### SUMMARY OF INVENTION

### Technical Problem

As described above, an OCT apparatus is demanded to have an optical system exhibiting a wider angle in order to enable collective acquisition of a tomographic image within a wider fundus range. In this case, the optical system of the OCT apparatus is optimally designed with a standard objective lens. Therefore, when wide angle imaging is required, such a measure is conceivable as to load the optical system by replacing the objective lens with a wide angle lens, or to insert an optical lens at a previous stage of the objective lens. Further, in the same manner, the OCT apparatus is further demanded to have an optical system exhibiting a narrower field angle in order to acquire the tomographic image within a narrower fundus range with high resolution power.

However, when an optical member (for example, wide angle lens) for changing the field angle exhibited by the optical system of the OCT apparatus is inserted into an optical path, values of various parameters deviate from suitable values. When a scanning speed of a scanning unit is assumed as a control parameter of a control portion of the OCT apparatus, for example, a resolution power of the image is lowered even in a case where the scanning speed remains fixed when the optical member is inserted. Further, when a dispersion compensation parameter is assumed as a signal processing parameter of a calculation processing portion of the OCT apparatus, for example, the inserted optical member causes dispersion of a measuring light, and hence the dispersion of the measuring light and dispersion of a reference light no longer match each other.

In view of the above-mentioned problems, the present invention has an object to acquire a preferred tomographic image of an object to be inspected by enabling values of various parameters to be switched to suitable values even when an optical member for changing a field angle is inserted in order to change the field angle of an imaging area of the tomographic image.

### Solution to Problem

In order to solve the above-mentioned problem, according to one embodiment of the present invention, there is provided an optical tomographic imaging apparatus, including:
a light source;
an optical splitter configured to split a light emitted from the light source into a measuring light and a reference light;
a scanning unit configured to scan an object to be inspected with the measuring light;
an optical system configured to irradiate the object to be inspected with the measuring light through the scanning unit;
a detector configured to receive an interference light between a return light of the measuring light from the object to be inspected and the reference light;
a calculation processing portion configured to process an output signal from the detector, to thereby acquire a tomographic image of the object to be inspected;
a determination unit configured to determine whether or not an optical member for changing a field angle is inserted between the scanning unit and the object to be inspected in order to change the field angle of an acquiring area of the tomographic image; and
a switching unit configured to switch a value of at least one parameter among a control parameter of a control portion configured to control the optical tomographic imaging apparatus, a signal processing parameter of the calculation processing portion, an image processing parameter, and an analysis processing parameter, based on a determination result from the determination unit.

### Advantageous Effects of Invention

According to the one embodiment of the present invention, a preferred tomographic image of the object to be inspected may be acquired by enabling the values of the various parameters to be switched to the suitable values even when the optical member for changing a field angle is inserted in order to change the field angle of an acquiring area of the tomographic image.

Further features of the present invention will become apparent from the following description of exemplary embodiments with reference to the attached drawings.

### BRIEF DESCRIPTION OF DRAWINGS

FIG. 1 is a diagram for schematically illustrating respective configurations included in an optical system of an ophthalmic apparatus according to one embodiment of the present invention.

FIGS. 2A, 2A′, 2A″, 2B, 2B′ and 2B″ are diagrams for illustrating: how an eye to be inspected is scanned with a measuring light in an x direction in the ophthalmic apparatus according to the one embodiment of the present invention; obtained two-dimensional fundus images; and obtained B-scan images, which are illustrated in respective cases of a usual field angle and a widened field angle.

FIG. 3A is a flowchart for illustrating a method of determining presence or absence of an insert lens on a measuring optical path by using an anterior ocular segment imaging portion in the ophthalmic apparatus illustrated in FIG. 1.

FIG. 3B is a flowchart for illustrating a method of determining the presence or absence of the insert lens on the measuring optical path by using an SLO portion in the ophthalmic apparatus illustrated in FIG. 1.

FIG. 3C is a flowchart for illustrating a method of determining the presence or absence of the insert lens on the measuring optical path by using an OCT portion in the ophthalmic apparatus illustrated in FIG. 1.

FIG. 4A is a flowchart for illustrating an entire process from acquisition of an OCT signal to an image analysis, which is conducted in the ophthalmic apparatus illustrated in FIG. 1.

FIG. 4B is a flowchart for illustrating an entire process from the acquisition of the OCT signal to the image analysis, which supports a wider field angle and is conducted in the ophthalmic apparatus illustrated in FIG. 1.

FIG. 4C is a flowchart for illustrating another entire process from the acquisition of the OCT signal to the image analysis, which supports the wider field angle and is conducted in the ophthalmic apparatus illustrated in FIG. 1.

FIG. 5A is a flowchart for illustrating a process of appropriately setting a resolution power being a control parameter under an imaging condition used when an OCT image is obtained.

FIG. 5B is a flowchart for illustrating a process of appropriately setting a parameter used when depth information is acquired, the parameter being a control parameter under an imaging condition used when the OCT image is obtained.

FIG. 5C is a flowchart for illustrating a process of appropriately setting a C-Gate position being a control parameter under an imaging condition used when the OCT image is obtained.

FIG. 6A is a flowchart for illustrating a process of appropriately setting a dispersion compensation parameter being a control parameter under an image composing condition used when the OCT image is obtained.

FIG. 6B is a flowchart for illustrating a process of appropriately setting a control parameter for obtaining an appropriate display distance under an image composing condition used when the OCT image is obtained.

FIG. 6C is a flowchart for illustrating a process of appropriately setting a control parameter when a map analysis is conducted under an image composing condition used when the OCT image is obtained.

FIG. 6D is a flowchart for illustrating a configuration for appropriately setting a control parameter when DOPU processing is conducted under an image composing condition used when the OCT image is obtained.

FIG. 6E is a flowchart for illustrating a configuration for appropriately setting a control parameter when blood speed processing is conducted under an image composing condition used when the OCT image is obtained.

FIGS. 7A and 7B are diagrams for illustrating examples of displaying a GUI when displaying the OCT image with the usual field angle and when displaying the OCT image with a wide field angle.

FIGS. 8A and 8B are diagrams for exemplifying modes of displaying, on the GUI, that the OCT image is obtained with the wider field angle.

FIG. 9 is a diagram for illustrating an imaging manner recommended when the OCT image with the wider field angle is obtained.

### DESCRIPTION OF EMBODIMENTS

Now, an embodiment of the present invention is described with reference to the accompanying drawings. Note that, the following embodiment is not intended to limit the present invention according to the scope of claims, and every combination of features described in this embodiment is not necessarily essential to the solution according to the present invention. Further, the description of the following embodiment is directed to an ophthalmic apparatus including a preferred optical tomographic (OCT) apparatus as an inspection apparatus according to the present invention.

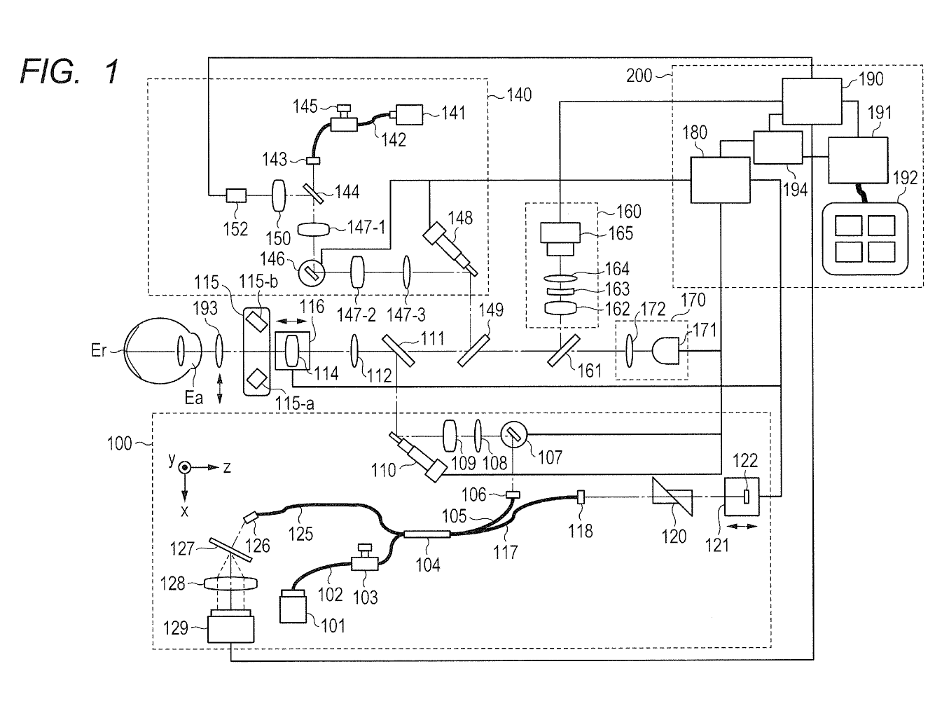

FIG. 1 is a schematic diagram of an overall configuration of the ophthalmic apparatus according to this embodiment.

This ophthalmic apparatus includes an optical tomographic (optical coherence tomography; hereinafter referred to as “OCT”) portion 100, a scanning ophthalmoscope (scanning laser ophthalmoscope; hereinafter referred to as “SLO”) portion 140, an anterior ocular segment observation portion 160, an internal fixation lamp portion 170, and a control portion 200. Note that, the control portion 200 may be formed integrally with the OCT portion 100, or may be separately formed as long as the control portion 200 and the OCT portion 100 are communicably connected to each other in a wired manner or in a wireless manner. For an actual inspection of an eye to be inspected, an illumination light source 115 described later and components such as optical members and the OCT portion 100 arranged in stages subsequent to the illumination light source 115 so as to be opposed to the eye to be inspected are received in a single casing, and are integrated as an optical head. When various kinds of imaging are conducted for the eye to be inspected as described later, the optical head executes an operation such as alignment for setting a distance from the eye to be inspected to an appropriate distance based on control of the control portion 200. In a state in which the eye to be inspected is caused to gaze at a fixation target by the internal fixation lamp portion 170, the alignment of the apparatus is conducted through use of an image of an anterior ocular segment of a subject observed by the anterior ocular segment observation portion 160. After completion of the alignment, a fundus of the eye to be inspected is imaged by the OCT portion 100 and the SLO portion 140. The respective configurations of this ophthalmic apparatus are described below.

### Configuration of OCT Portion 100

Now, the configuration of the OCT portion 100 is described with reference to FIG. 1.

A light source 101 is a super luminescent diode (SLD) light source being a low-coherence light source, and emits, for example, a light having a central wavelength of 850 nm and a bandwidth of 50 nm. Note that, the SLD light source is used as the light source 101 in this embodiment, but any light source capable of emitting a low-coherence light, such as an amplified spontaneous emission (ASE) light source, may be used.

The light emitted from the light source 101 is guided to a fiber coupler 104 through a fiber 102 and a polarization controller 103, to be branched off into a measuring light (referred to also as “OCT measuring light”) and a reference light. The polarization controller 103 is configured to adjust a state of polarization of the light emitted from the light source 101, and in this case, the light is adjusted to be linearly polarized. A branching ratio of the fiber coupler 104 used in this embodiment is (90 (reference light)):(10 (measuring light)).

The branched-off measuring light is emitted as a parallel light from a collimator 106 through a fiber 105. The emitted measuring light reaches a dichroic mirror (DCM) 111 through an X scanner 107, a lens 108, a lens 109, and a Y scanner 110. Note that, the X scanner 107 is formed of a galvanometer mirror configured to scan a fundus Er with the measuring light in a horizontal direction, and the Y scanner 110 is formed of a galvanometer mirror configured to scan the fundus Er with the measuring light in a vertical direction. Further, the X scanner 107 and the Y scanner 110 that form a scanning unit are controlled by a drive control portion 180, and can scan a region on the fundus Er within a desired range with the measuring light. In this case, it is preferred that the scanning unit be arranged at a position conjugate with the anterior ocular segment of the eye to be inspected, to scan the fundus with the measuring light. At this time, vignetting of the measuring light in the anterior ocular segment can be reduced. Further, the DCM 111 has a characteristic of reflecting a light of from 800 nm to 900 nm and transmitting a light other than the light of from 800 nm to 900 nm.

The measuring light reflected by the DCM 111 passes through a lens 112, a focus lens 114, and an anterior ocular segment Ea to irradiate a retinal layer of the fundus Er. The measuring light is focused on the retinal layer of the fundus Er by the focus lens 114 supported by a stage 116 so as to be movable in an optical axis direction. The movement of the focus lens 114 in the optical axis direction is controlled by the drive control portion 180. The measuring light that has irradiated the fundus Er is scattered and reflected by each retinal layer, and returns to the fiber coupler 104 while following back the optical path described above.

On the other hand, the reference light branched off by the fiber coupler 104 is emitted as a parallel light from a collimator 118 through a fiber 117. The emitted reference light is reflected by a mirror 122 on a coherence gate stage 121 through dispersion compensation glass 120, and returns to the fiber coupler 104. The coherence gate stage 121 has the mirror 122 controlled to move in the optical axis direction by the drive control portion 180 so as to handle a difference in an ocular axial length of a subject or the like. This allows control of an optical path length difference between an optical path length of the measuring light and an optical path length of the reference light.

The measuring light and the reference light that have returned to the fiber coupler 104 are multiplexed to become an interference light. The above-mentioned optical path length difference is suitably controlled, to thereby obtain the interference light capable of generating a preferred OCT signal. The interference light is guided to a grating 127 through a fiber 125 and a collimator 126, dispersed by the grating 127, and then received by a line camera 129 through a lens 128. The light received by the line camera 129 is set as an electric signal corresponding to an intensity of the light, and output to a signal processing portion 190.

In the configuration described above, the fiber coupler 104 corresponds to an optical splitter configured to split the light emitted from the light source 101 into the measuring light and the reference light, and the configuration of a scanner or the like arranged in an optical path of the OCT portion 100 corresponds to an optical system configured to irradiate the eye to be inspected with the measuring light. Further, the line camera 129 corresponds to a detector configured to receive the interference light between a return light of the measuring light from the eye to be inspected and the reference light. In addition, the signal processing portion 190 corresponds to a calculation processing portion configured to execute signal processing, image processing, and analysis processing for an output signal corresponding to the interference light received from the line camera 129, to thereby acquire a tomographic image of the eye to be inspected.

### Configuration of SLO Portion 140

Next, an example of the configuration of the SLO portion 140 is described with reference to FIG. 1.

Note that, in this embodiment, the SLO portion 140 corresponds to an example of a fundus image acquisition unit configured to acquire a fundus image of the eye to be inspected.

A light source 141 is, for example, a semiconductor laser, and in this embodiment, emits a light having a central wavelength of 780 nm as the measuring light. The measuring light (referred to also as “SLO measuring light”) emitted from the light source 141 is adjusted to be linearly polarized by the polarization controller 145 after passing through a fiber 142, and emitted as a parallel light from a collimator 143. The emitted measuring light passes through a holed portion of a holed mirror 144 to reach an X scanner 146 through a lens 147-1. The X scanner 146 is formed of a galvanometer mirror configured to scan the fundus Er with the measuring light in the horizontal direction. The measuring light that has passed through the X scanner 146 reaches a Y scanner 148 through a lens 147-2 and a lens 147-3. The Y scanner 148 is formed of a galvanometer mirror configured to scan the fundus Er with the measuring light in the vertical direction. The measuring light that has passed through the Y scanner 148 reaches a second dichroic mirror (DCM) 149. Note that, the polarization controller 145 may be omitted. The X scanner 146 and the Y scanner 148 are controlled by the drive control portion 180 described later, and scan the fundus within the desired range with the measuring light. The second DCM 149 has a characteristic of reflecting a light of, for example, from 760 nm to 800 nm and transmitting a light other than the light of from 760 nm to 800 nm.

The linearly polarized measuring light reflected by the second DCM 149 passes through the DCM 111, and then passes along the same optical path as the OCT measuring light from the OCT portion 100, to reach the fundus Er.

The SLO measuring light that has irradiated the fundus Er is scattered and reflected by the fundus Er, and reaches the holed mirror 144 while following back the above-mentioned optical path. The light reflected by the holed mirror 144 is received by an avalanche photodiode (hereinafter referred to as “APD”) 152 through a lens 150, converted into an electric signal, and output to the signal processing portion 190 described later.

In this case, the position of the holed mirror 144 is conjugate with a pupil position of the eye to be inspected, and among lights obtained after the measuring light applied to the fundus Er is scattered and reflected, the light that has passed through a periphery of a pupil is reflected by the holed mirror 144. Note that, in this embodiment, the holed mirror 144 is used to separate the optical path, but the present invention is not limited thereto, and, for example, a prism onto which a hollow mirror has been evaporated may be used for this configuration.

### Configuration of Anterior Ocular Segment Observation Portion 160

Next, the configuration of an anterior ocular segment observation portion 160 is described with reference to the accompanying drawings.

The anterior ocular segment observation portion 160 images the anterior ocular segment Ea illuminated by the illumination light source 115 formed of an LED 115-a and an LED 115-b configured to emit an illumination light having a wavelength of 1,000 nm. The light applied by the illumination light source 115 and reflected by the anterior ocular segment Ea passes through the focus lens 114, the lens 112, the DCM 111, and the second DCM 149 to reach a third DCM 161. The third DCM 161 has a characteristic of reflecting a light of from 980 nm to 1,100 nm and transmitting a light other than the light of from 980 nm to 1,100 nm. The light reflected by the third DCM 161 passes through a lens 162, a lens 163, and a lens 164, and is received by an anterior ocular segment camera 165. The light received by the anterior ocular segment camera 165 is converted into an electric signal, and output to the signal processing portion 190.

### Configuration of Internal Fixation Lamp Portion 170

Next, the configuration of the internal fixation lamp portion 170 is described with reference to the accompanying drawings.

The internal fixation lamp portion 170 includes a display portion 171 and a lens 172. As the display portion 171, a plurality of light emitting diodes (LDs) arranged in a matrix shape are used. A lit position of the light emitting diode is changed depending on a site to be imaged under control of the drive control portion 180. The light from the display portion 171 is guided to the eye to be inspected through the lens 172. The light emitted from the display portion 171 is of 520 nm, and a desired pattern is displayed by the drive control portion 180. The internal fixation lamp portion 170 promotes fixation by causing the subject to gaze at the lit position on the display portion 171, and the imaging of the eye to be inspected is executed in such a state, to thereby obtain the image of a part to be imaged.

### Configuration of Control Portion 200

The configuration of the control portion 200 is described with reference to the accompanying drawings.

The control portion 200 includes the drive control portion 180, the signal processing portion 190, a display control portion 191, a display portion 192, and a switching portion 194. Note that, the display portion 192 may be separately formed as long as the display portion 192 is communicably connected to the control portion 200.

As described above, the drive control portion 180 controls the X scanner 107, the Y scanner 110, the X scanner 146, the Y scanner 148, the coherence gate stage 121, the focus lens stage 116, and the display portion 171. Further, the drive control portion 180 controls respective portions such as the drive system for the alignment of the optical head formed of the casing including the OCT portion 100 with reference to the eye to be inspected.

The signal processing portion 190 generates an image, analyzes the generated image, or generates visualization information on an analysis result based on a signal output from each of the line camera 129, the APD 152 described later, and the anterior ocular segment camera 165. Note that, generation of the image and the like is described later in detail.

The display control portion 191 displays the image generated by the signal processing portion 190 and the like on a display screen of the display portion 192. Under control of the display control portion 191 configured to specify display contents or the like, the display portion 192 displays various kinds of information as described later. Further, the switching portion 194 includes a module area that functions as a switching unit configured to control the entire apparatus and switch at least one of control parameters of control portions such as the drive control portion 180 and the display control portion 191 and respective processing parameters used when the OCT signal is processed by the signal processing portion 190. Note that, the respective processing parameters include a signal processing parameter such as a gain, an image processing parameter used when the image processing is executed to generate the image, and an analysis parameter used when an image analysis such as map processing described later is executed.

### Tomographic Image Generation and Fundus Image Generation

Next, each processing of the image generation and the image analysis executed by the signal processing portion 190 is described.

The signal processing portion 190 subjects an interference signal output from the line camera 129 to reconstruction processing used for a general spectral domain OCT (SD-OCT), to thereby generate the tomographic image based on each polarization component. First, the signal processing portion 190 removes the fixed pattern noise from the interference signal. The removal of the fixed pattern noise is conducted by averaging a plurality of A-scan signals that have been detected to extract a fixed pattern noise and subtracting the fixed pattern noise from the input interference signal. Subsequently, the signal processing portion 190 converts the interference signal from a wavelength into a wave number, and then conducts a Fourier transform therefor, to thereby generate a tomographic signal.

The signal processing portion 190 also processes reflected light intensity information for the signal output from the APD 152, to thereby generate the fundus image.

### Changing of Field Angle

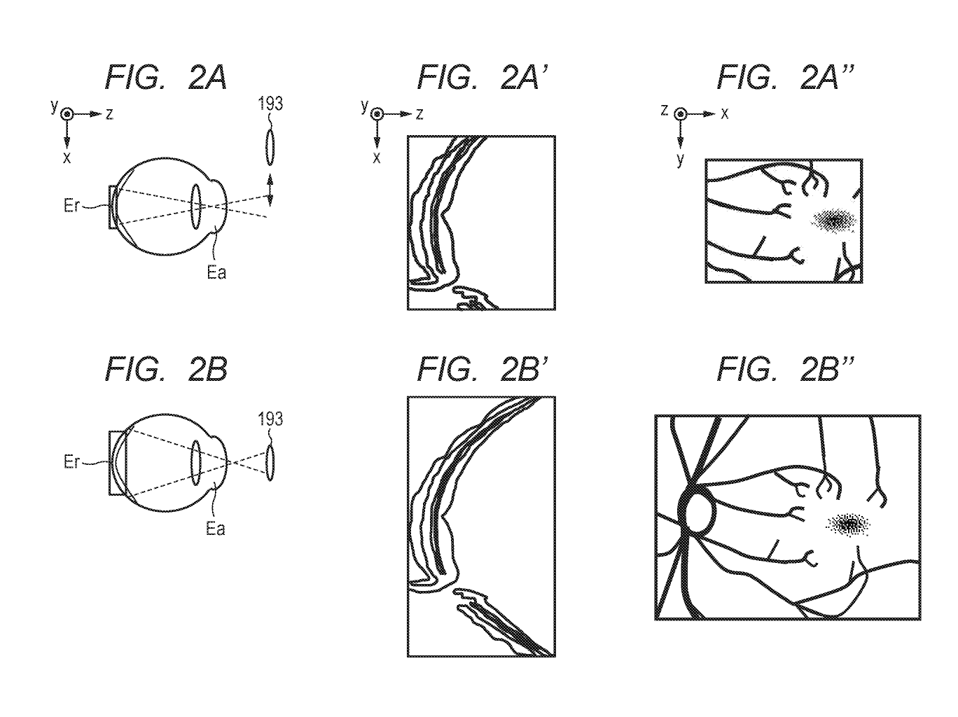

Next, a case where such an apparatus as described above is used to image the image of a fundus (Er) with a changed field angle is described. In this embodiment, as a configuration for changing an image acquiring area within the fundus image, an insert lens 193 is inserted as an adapter lens between the eye to be inspected and the optical head. FIG. 2A, FIG. 2A′, FIG. 2A″, FIG. 2B, FIG. 2B′, and FIG. 2B″ are diagrams for schematically illustrating a scanning range of the measuring light based on the presence or absence of the insert lens 193 within a cross section of the eye to be inspected. The insert lens 193 is inserted into the optical path of the measuring light, to thereby change the optical path so as to change the scanning range from the scanning range indicated by the broken lines in FIG. 2A to the scanning range indicated by the broken lines in FIG. 2B. This widens the scanning range of the measuring light on the fundus (Er), and allows the fundus to be imaged with a larger region (hereinafter referred to as “wide field angle”).

Specifically, OCT images within a range between a field angle illustrated in FIG. 2A′ and a field angle illustrated in FIG. 2B′ can be acquired. Note that, in a case of conducting the OCT imaging with a wide field angle, it is preferred that a depth-direction imaging range be set longer than a depth-direction imaging range of the OCT image of a usual field angle so that the curved fundus falls within an imaging range as much as possible. Further, SLO images within a range between a field angle illustrated in FIG. 2A″ and a field angle illustrated in FIG. 2B″ can also be acquired. In this embodiment, a lens of −20 D is used as the insert lens 193 to achieve the wide field angle. When the lens of −20 D is used as the insert lens 193, the field angle becomes approximately 1.5 times as large as that of an original image. In this embodiment, the imaging range or the image acquiring area is widened from 10 mm to 15 mm in terms of an x-direction scanning distance of the OCT image. Note that, the field angle is set to 1.5 times in this embodiment, but it should be understood that this magnification is merely an example based on an eyeglass, use of which is assumed in this embodiment, and may be a variable value.

Note that, the use of the eyeglass as the insert lens 193 is assumed in the above-mentioned embodiment, but the configuration that can support the insert lens 193 is not limited thereto. A contact lens, an adapter lens mounted on the ophthalmic apparatus, or other such optical members that can be inserted into a measuring optical path for changing the field angle may be employed as an insert lens therefor as long as the insert lens is removably inserted between the scanning unit within an OCT apparatus and the eye to be inspected and can change the field angle. Further, this embodiment may be applied not only to insertion of the optical member for a wider field angle but also to insertion of an optical member for a narrower field angle.

### Overall Flow

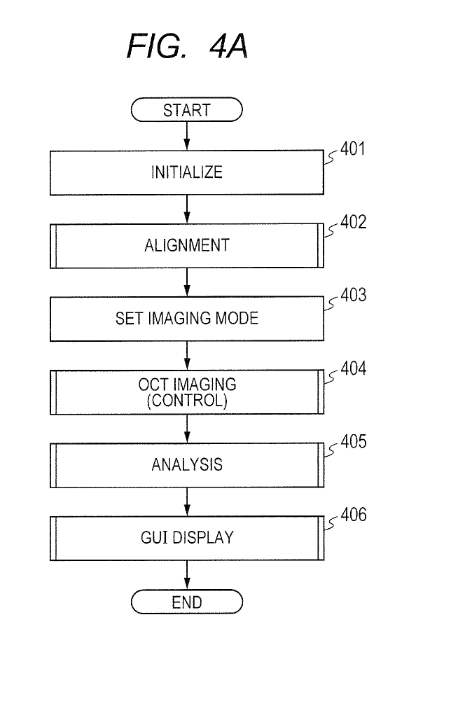

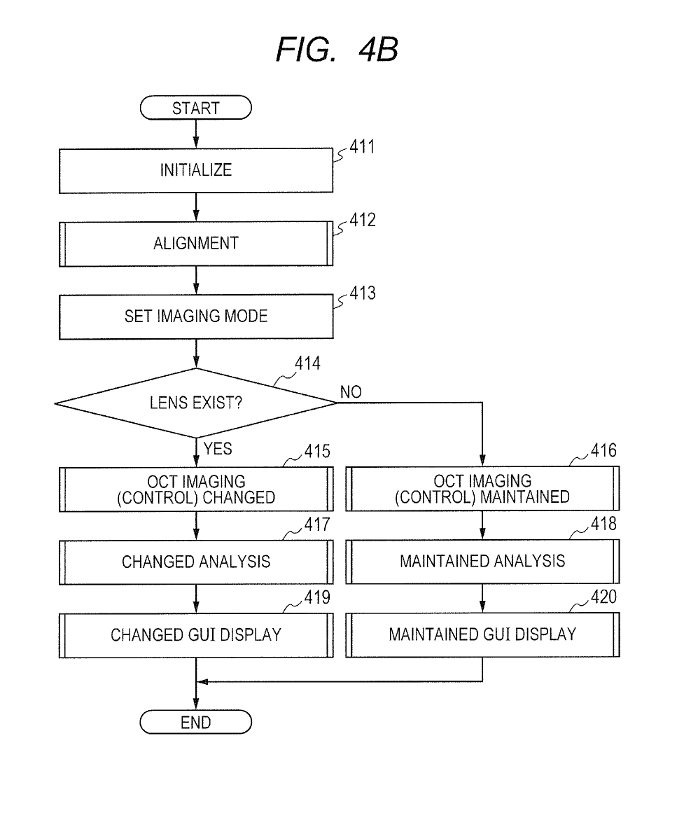

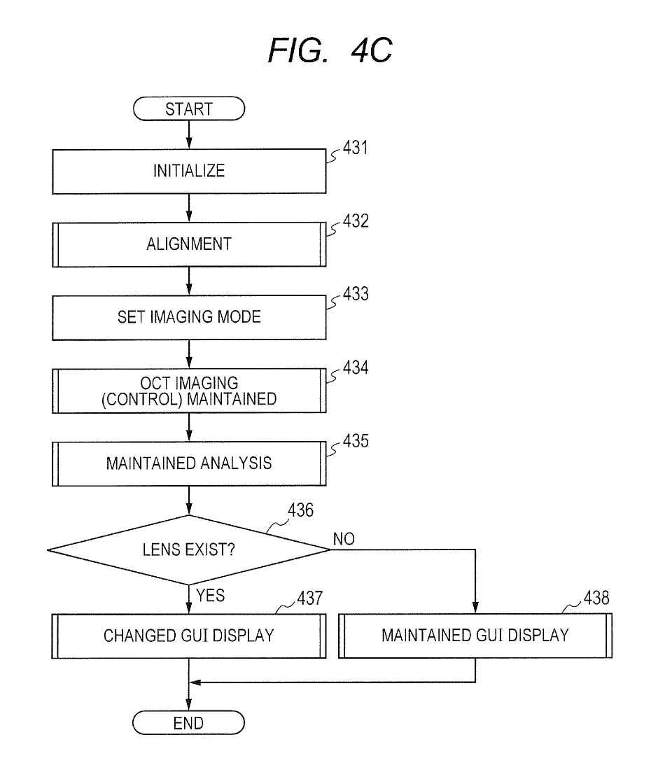

An overall flow from the imaging of the OCT image to outputting of an analysis screen conducted by using the above-mentioned ophthalmic apparatus is described with reference to flowcharts illustrated in FIG. 4A, FIG. 4B, and FIG. 4C.

First, a process that leads to the outputting of the OCT image of the usual field angle is described with reference to the flowchart illustrated in FIG. 4A. In a case of imaging an OCT image, the ophthalmic apparatus conducts initialization (such as electrical check, safety check for a light amount or the like, and mechanical check) (Step 401). After the initialization is finished, the alignment (adjustment of a distance between the subject and a main body, focus adjustment, C-gate adjustment, and fixation adjustment) of the ophthalmic apparatus is conducted (Step 402). After that, an imaging mode (such as a macula mode or a glaucoma mode) is set (Step 403), and the OCT imaging (control) in the set mode is conducted (Step 404), to thereby acquire the image signal. Subsequently, the signal processing for the obtained image signal is conducted to acquire the OCT image, and the OCT image is analyzed (Step 405). Simultaneously or after that, a result thereof is displayed on a display (GUI display) (Step 406).

Next, a specific example of this embodiment in the case of changing the field angle is described below. In this embodiment, a case of automatically detecting the insert lens 193 at a time of the alignment (Step 402) in the above-mentioned flowchart so as to cause the subsequent process to support a wide field angle is described. In other words, an overall flow of an example in which the OCT imaging (control), the analysis, and the GUI display are conducted after a wider field angle is supported is described.

First, in the same manner as in the case of the overall flow illustrated in FIG. 4A, the initialization is conducted (Step 411). Subsequently, the alignment is conducted (Step 412), to thereby determine the presence or absence of the insert lens 193. Note that, a method for this determination is described later. Subsequently, after the imaging mode is set (Step 413), imaging control for the OCT image is changed based on the presence or absence of the insert lens 193 on the optical path (Step 414). Specifically, when it is determined that the insert lens 193 exists, the flow advances to Step 415, and when it is determined that the insert lens 193 does not exist, the flow advances to Step 416. Examples of the parameter to be controlled at a time of OCT image imaging include a scanning speed of the scanner and a step interval of the scanner, and setting values of those parameters are changed. Note that, the changing of the control parameter of the control portion is described later.

In addition, the analysis condition is changed based on the presence or absence of the insert lens 193. Specifically, when it is determined that the insert lens 193 exists, the flow advances from Step 415 to Step 417. Further, when it is determined that the insert lens 193 does not exist, the flow advances from Step 416 to Step 418. The analysis condition to be changed is exemplified by, for example, a calculation condition for a macula-papilla distance.

After that, the GUI display (Step 419 and Step 420) is also set appropriately based on the presence or absence of the insert lens 193 on the optical path. A display condition to be changed is exemplified by, for example, an image display position to be changed.

Note that, in the example illustrated in FIG. 4B, it is assumed that the presence or absence of the insert lens 193 on the optical path is automatically determined, and that the flow is also automatically determined in turn. However, for example, to meet a demand for a speedup in the processing that leads to the display or so-called usability of the apparatus, a part to be changed may be reduced in number on purpose through a user's setting or the like. Such an example is illustrated in FIG. 4C. In a flowchart of FIG. 4C, the process from the initialization of Step 431 to the analysis of Step 435 is the same as the process from Step 401 to Step 405 in FIG. 4A. The presence or absence of the insert lens 193 on the optical path is reflected only in a condition used when the GUI display is conducted in Step 436. After the determination of the presence or absence of the insert lens 193, an operation of Step 419 or Step 420 in FIG. 4B is executed in Step 437 or Step 438.

### Determination Method for Presence or Absence of Insert Lens 193: Use of Various Images or Signals

Now, a determination method for the presence or absence of the insert lens 193 on the measuring optical path is described. In this embodiment, an object is achieved without new addition of a detection apparatus for the insert lens 193. Note that, an example in which the insert lens 193 is detected during the alignment (Step 402) of the apparatus in the above-mentioned overall flow is described in this section. In addition, an example in which the insertion of the insert lens 193 into the measuring optical path is detected when an inspector puts a check mark on a GUI screen is described. Note that, the determination of the presence or absence of the insertion of the insert lens 193, which is provided as an optical member for changing a field angle described later, into the measuring optical path is executed by a module area that functions as a determination unit in the switching portion 194. Further, a determination result from the determination unit may define a determination criterion for the parameter to be switched by the above-mentioned switching unit. Note that, the module area that functions as a determination unit may be formed as a determination portion (not shown) provided separately from the switching portion 194.

### (1) Determination of Presence or Absence of Insert Lens 193 Based on Analysis Result of Anterior Ocular Segment Image

As specific determination processing, the anterior ocular segment observation portion 160 is used to determine the presence or absence of the insert lens 193 based on a reflected light of an anterior ocular segment imaging light. At a time of an actual inspection of the eye to be inspected, the anterior ocular segment imaging light is reflected by a front surface or a back surface of the insert lens 193. The anterior ocular segment camera 165 can receive the reflected light. The presence or absence of the insert lens 193 on the optical path is determined based on whether or not the reflected light has been received, and the determination result is stored into a memory (not shown).

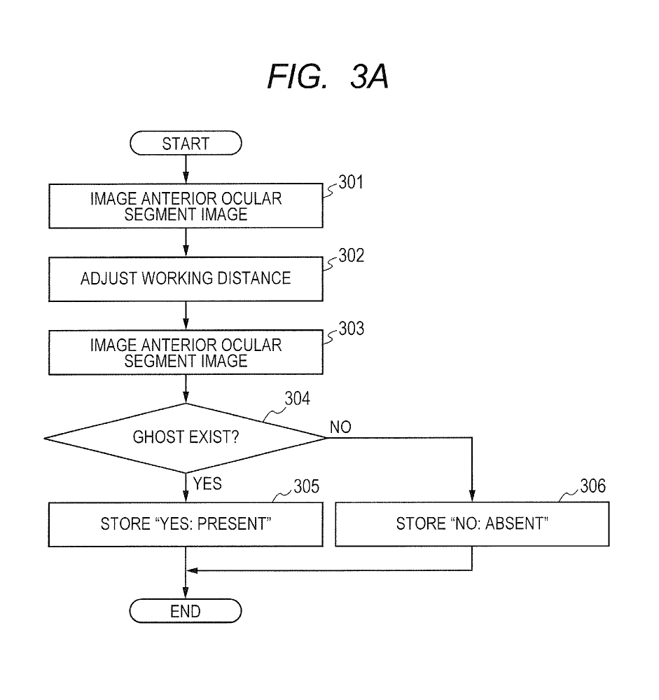

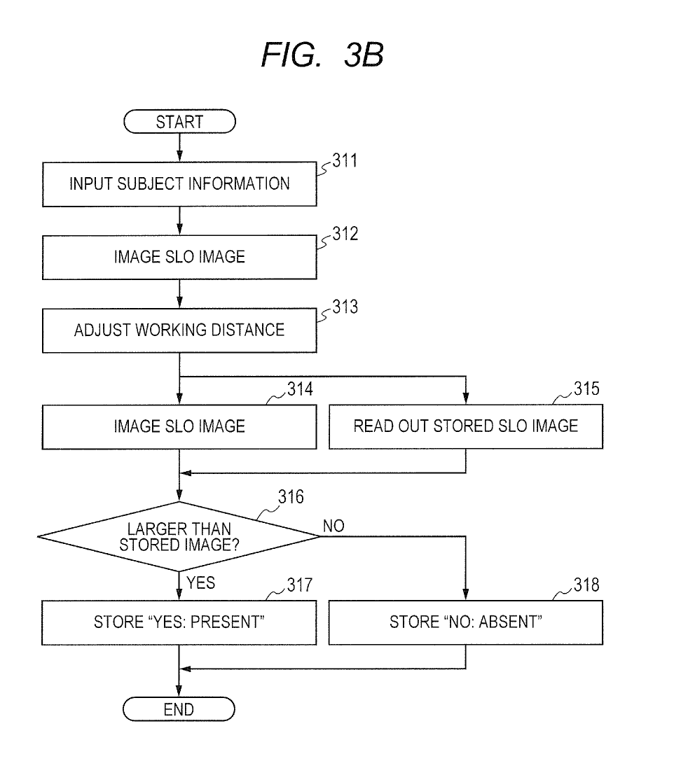

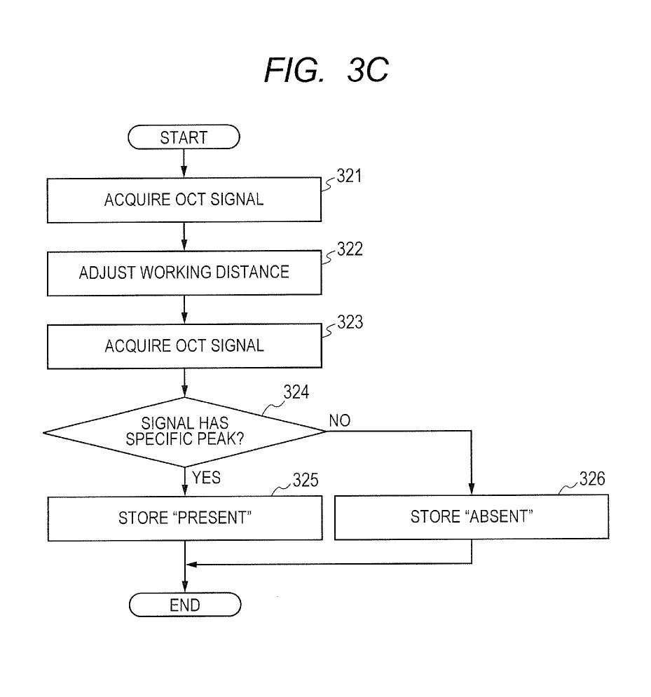

A specific flow of this determination method is illustrated in FIG. 3A. First, an anterior ocular segment image is acquired (Step 301), and then the distance (working distance) between the eye to be inspected and the main body is adjusted (Step 302). After the adjustment, the anterior ocular segment image is acquired again (Step 303). After the anterior ocular segment image is acquired again, the image analysis is executed to determine whether or not the reflected light (ghost) of the insert lens 193 exists in the anterior ocular segment image acquired in Step 303 (Step 304). Any result (presence or absence of the ghost) of the image analysis that has been obtained is stored into the memory (not shown) (Step 305 and Step 306).

### (2) Determination of Presence or Absence of Insert Lens 193 Based on Analysis Result of Fundus Image Obtained by SLO Portion

The presence or absence of the insert lens 193 may also be determined by a configuration other than the anterior ocular segment observation portion 160. Next, an example in which the SLO portion 140 is used to execute the determination of the presence or absence of the insert lens 193 is described. In this case, data on the anterior ocular segment image obtained in the past is compared with data on the anterior ocular segment image obtained immediately before by the SLO portion 140, to thereby determine the presence or absence of the insert lens 193 on the measuring optical path. Specifically, it is assumed that the presence or absence of the insert lens 193 is determined based on a distance (pixel number) between a center of a macula and the blood vessel, and that the determination result is stored into the memory (not shown).

A specific flow of this determination method is illustrated in FIG. 3B. First, subject information is input (Step 311), the SLO image is acquired (Step 312), and then a focus of the SLO portion 140 is adjusted with respect to the fundus of the eye to be inspected for focusing for obtaining the image (Step 313). After the adjustment, the SLO image is acquired again (Step 314). After the SLO image is acquired again, based on the subject information input in Step 311, the SLO image obtained in the past is read out from the memory (not shown) or a database (not shown). Note that, the database is communicably connected to the control portion 200 in a wired manner or in a wireless manner, and allows a search to be made based on an input ID of the subject for the data obtained in the past associated with the ID, to read out the retrieved data. An image comparison is made between the SLO image obtained in the past and the SLO image acquired again in Step 314 (Step 316). The presence or absence of a change in the image is determined based on the comparison between those images, and any determination result that has been obtained is stored into the memory (not shown) (Step 317 and Step 318).

The SLO portion 140 and the anterior ocular segment observation portion 160 according to this embodiment that are described above form a second detection portion configured to receive the return light from the eye to be inspected in order to acquire at least one of the anterior ocular segment image of the eye to be inspected or the fundus image of the eye to be inspected. The above-mentioned determination unit within the switching portion 194 can determine whether or not the insert lens 193 has been inserted into the measuring optical path based on the output signal from the second detection portion.

### (3) Determination of Presence or Absence of Insert Lens 193 Based on OCT Signal

Further, the presence or absence of the insert lens 193 may also be determined by a configuration other than the anterior ocular segment observation portion 160 or the SLO portion 140 described above. Next, an example in which the OCT portion 100 is used to execute the determination of the presence or absence of the insert lens 193 on the measuring optical path is described. Specifically, when the insert lens 193 is inserted into the measuring optical path, the signal of the reflected light due to the insert lens 193 is observed in the OCT signal that has undergone FFT processing. In this case, the presence or absence of the insert lens 193 is determined based on the presence or absence of the ghost corresponding to the signal of the reflected light. In other words, in this mode, the above-mentioned determination unit determines whether or not the insert lens 193 has been inserted into the measuring optical path based on the output signal from the line camera 129 provided as the detector.

A specific flow of this determination method is illustrated in FIG. 3C. First, the OCT signal is acquired (Step 321), and then a C-Gate position is adjusted so as to allow the OCT image to be acquired (Step 322). After the adjustment, the OCT signal is acquired again (Step 323). In addition, the OCT signal acquired in Step 323 is analyzed (Step 324). The presence or absence of the ghost is determined as a result of the analysis, and any result that has been obtained is stored into the memory (not shown) (Step 325 and Step 326).

### (4) Other Examples Relating to Determination of Presence or Absence of Insert Lens 193

The determination methods for the insert lens 193 are described above, but the determination method is not limited thereto. For example, an anterior ocular segment monitor may be used to determine the presence or absence of the insert lens 193 on the measuring optical path by making a comparison with the data obtained in the past (in terms of a pupil diameter or the like) and further executing the signal processing for the image (in terms of a luminance distribution) or the like. Further, the SLO portion 140 may be used to determine the presence or absence of the insert lens 193 on the measuring optical path by executing the determination of the presence or absence of the ghost in the SLO image (such as a binarization region analysis using a gamma ray), acquisition of a signal intensity distribution, calculation of the macula-papilla distance, or the like.

Further, the OCT portion 100 may be used to determine the presence or absence of the insert lens 193 on the measuring optical path by executing detection of the ghost in the OCT image, generation of a pseudo SLO ghost image from the OCT signal, the comparison with the data obtained in the past, an analysis of a graph representing a decrease in an OCT sensitivity, or the like. Note that, in the detection of the ghost in the OCT image, it is preferred that an area detection of a high-luminance region or the like be conducted for the B-scan image. Further, the pseudo SLO ghost image is generated by analyzing a C-scan image generated from the OCT signal. In the comparison with the data obtained in the past, it is preferred that the comparison be made with the B-scan image or with the C-scan image. Further, the graph representing the decrease in the OCT sensitivity is analyzed on the assumption that the graph includes information on a decrease in a sensitivity due to insertion of a lens.

Further, another new mechanism may be provided such as an input (such as a switch or a GUI input) to be made by the user or another unit (magnetic one) for detecting the lens. The same effects are produced even when such a mechanism is used to determine the presence or absence of the insert lens 193 on the measuring optical path. In other words, the presence or absence of the insertion of the insert lens 193 onto the measuring optical path may be determined by providing an input unit configured to input the presence or absence by an operator. In this case, the above-mentioned determination unit determines that the insert lens 193 has been inserted into the measuring optical path based on the input made through the input unit.

Note that, this detection mechanism is assumed to mainly target a case where eyeglasses exist on the measuring optical path as the insert lens 193 as described above. Therefore, when an OCT attachment for an anterior ocular segment is used, it is preferred that, in order to distinguish between the eyeglasses and the attachment, a different detection mechanism be provided separately from the above-mentioned existing configuration of the ophthalmic apparatus. Such a detection mechanism is provided to thereby allow sensing of an accurate power of the insert lens 193.

### Changing of OCT Imaging (Control) Condition: Switching of Control Parameter of Control Portion

Next, the switching of the control parameter of the control portion such as the drive control portion 180 or the display control portion 191 of the OCT apparatus is described.

### (1) Control Parameter 1: Switching of Scanning Speed of Scanning Unit

As illustrated in FIG. 2A, FIG. 2A′, FIG. 2A″, FIG. 2B, FIG. 2B′, and FIG. 2B″, the insertion of the insert lens 193 into the measuring optical path allows a wide-field-angle OCT image to be acquired. However, the wide-field-angle OCT image illustrated in FIG. 2B′ has a lower resolution power (in an x direction in FIG. 2A, FIG. 2A′, FIG. 2A″, FIG. 2B, FIG. 2B′, and FIG. 2B″) than the OCT image illustrated in FIG. 2A′. This is because an imaging time period is the same irrespective of an increased field angle (imaging distance), and signals are thinned out, to thereby lower the resolution power. In this embodiment, in order to prevent the resolution power from being lowered, the scanning speed of the X scanner 107 of the OCT portion 100 is lowered to the scanning speed 1/1.5 times as large as usual (because the field angle becomes 1.5 times larger), to thereby acquire the image having the resolution power that is not lowered.

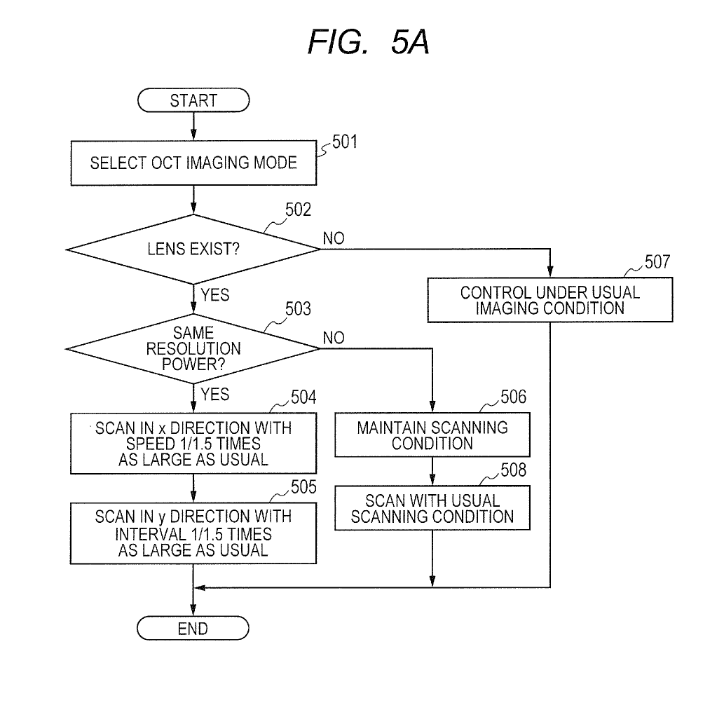

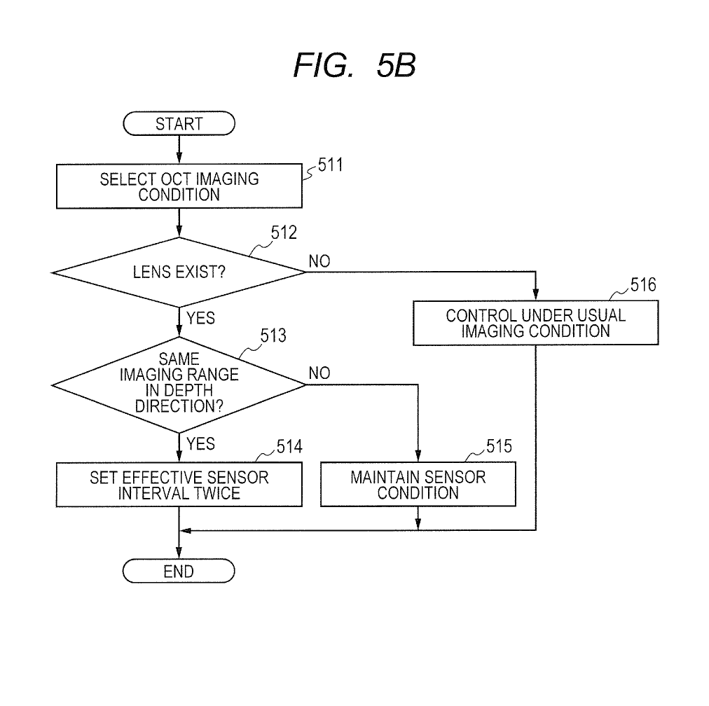

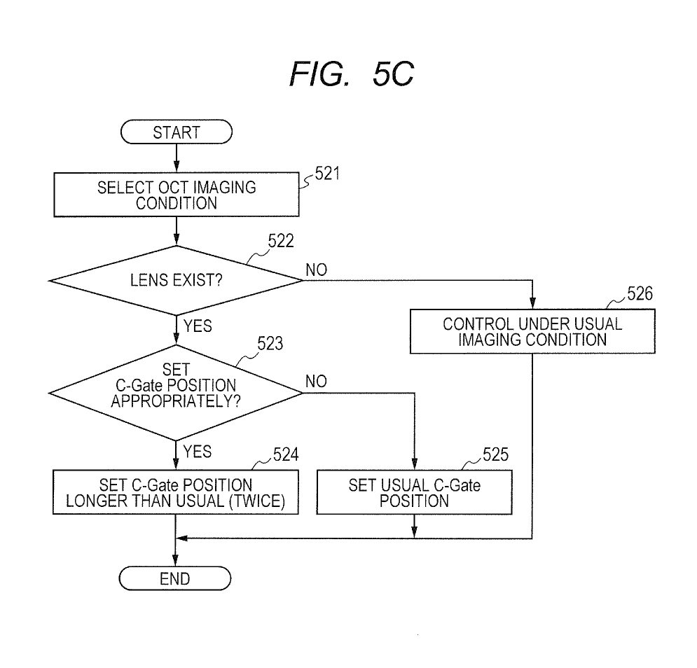

A specific process for handling such lowering of a resolution power is described below with reference to a flowchart illustrated in FIG. 5A. First, the OCT imaging mode is selected (Step 501), and then the information on the presence or absence of the insert lens 193 on the measuring optical path is obtained (Step 502). When it is determined in Step 502 that the insert lens 193 is inserted in the measuring optical path, the flow advances to Step 503. In Step 503, it is displayed, on a GUI, whether or not to set the resolution power to be the same, and the user is caused to make a selection thereof. When the setting of the resolution power to be the same is selected, the flow advances to Step 504, where the X scanner 107 is operated with a speed 1/1.5 times as large as usual (because the field angle becomes 1.5 times larger). Further, the Y scanner 110 is operated with an interval 1/1.5 times as large as usual (because the field angle becomes 1.5 times larger) in the same manner (Step 505), to thereby acquire the OCT image having the same resolution power in the x direction and a y direction. When it is determined in Step 502 that the insert lens 193 does not exist on the optical path, the flow advances to Step 507, where the OCT image of the usual field angle is imaged. Further, when the setting of the resolution power to be the same is not selected in Step 503, it is determined that the lowered resolution power is wished (Step 506), and the imaging of the OCT image is executed with only the field angle changed while the scanning condition of the scanner is maintained (Step 508).

The scanning speed of an X scanner and a Y scanner, which form the scanning unit configured to scan the eye to be inspected with the measuring light described above, is an example of the control parameter according to this embodiment, and the above-mentioned switching unit switches the scanning speed when the insert lens 193 is inserted into the measuring optical path.

### (2) Control Parameter 2: Switching of Sensor Interval of Line Camera

Now, among the OCT apparatus, there also exists one that has a mechanism capable of variably setting an effective pixel number of the line camera 129. Such an apparatus allows an appropriate image to be acquired by setting a mode capable of obtaining depth information indicating a larger depth depending on the insertion of the insert lens 193 into the measuring optical path. The appropriate image referred to herein represents, for example, an image exhibiting no image fold and having the same X-Z ratio as the OCT image of the usual field angle.

A process of acquiring such an OCT image is described with reference to a flowchart illustrated in FIG. 5B. In this process, the OCT imaging mode is first selected (Step 511), and then the information on the presence or absence of the insert lens 193 on the measuring optical path is obtained (Step 512). When it is determined in Step 512 that the insert lens 193 is inserted in the measuring optical path, the flow advances to Step 513. In Step 513, it is displayed, on the GUI, whether or not to set the depth-direction imaging range to be the same, and the user is caused to make a selection thereof. When the setting of the depth-direction imaging range to be deeper is selected, the flow advances to Step 514, where the sensor interval of the line camera 129 is changed (signal acquisition interval: ½ times; sensor number per unit length: twice). Further, in the same manner as in the case illustrated in FIG. 5A, when it is determined in Step 512 that the insert lens 193 does not exist on the measuring optical path, the flow advances to Step 516, where the imaging of the OCT image is executed under a usual imaging condition. In addition, when the setting of the depth-direction imaging range not to be changed is selected in Step 513, the flow advances to Step 515, where the imaging of the OCT image is executed with only the field angle changed while the control of a line camera is maintained.

### (3) Control Parameter 3: Switching of Coherence Gate Position

Now, it is conceivable that a widened field angle causes the above-mentioned image fold or a decrease in a signal intensity at a site to be observed. Accordingly, in order to suppress those phenomena, a coherence gate (C-Gate) position is required to be set appropriately.

A specific process for handling such phenomena that can be caused by the widening of the field angle is described below with reference to a flowchart illustrated in FIG. 5C. In this process, the OCT imaging mode is first selected (Step 521), and then the information on the presence or absence of the insert lens 193 on the measuring optical path is obtained (Step 522). When it is determined in Step 522 that the insert lens 193 is inserted in the measuring optical path, the flow advances to Step 523. In Step 523, it is displayed, on the GUI, whether or not to set the C-Gate position appropriately, and the user is caused to make a selection thereof. When the appropriately setting of the C-Gate position is selected, the flow advances to Step 524. Now, a consideration is given to a distance between a surface of a retina and the C-Gate position in a central vicinity of the imaging range in the horizontal direction. The C-Gate position is set so that the distance exhibited when the insert lens 193 is inserted becomes longer than (for example, at least two times as long as) the distance exhibited at the time of the OCT imaging with a usual field angle. At this time, as described above, when the OCT imaging is conducted with a wide field angle, it is preferred that the depth-direction imaging range be set longer than the depth-direction imaging range of the OCT image of the usual field angle so that the curved fundus falls within the imaging range as much as possible (see FIG. 2B′). Further, in the same manner as in the case illustrated in FIG. 5A, when it is determined in Step 522 that the insert lens 193 does not exist on the measuring optical path, the flow advances to Step 526, where the imaging of the OCT image is executed under the usual imaging condition. In addition, when the setting of the C-Gate position not to be changed is selected in Step 523, the imaging of the OCT image is executed with only the field angle changed while the control of the C-Gate position is maintained. The above-mentioned appropriately setting of the C-Gate position includes the setting of the C-Gate position on a choroid side.

### (4) Control Parameter 4: Others

Note that, this embodiment is described by taking the above-mentioned three examples of the control regarding resetting of the control condition involved in the changing of the field angle. However, a manner of the resetting of the control condition is not limited to those forms. For example, it should be understood that an optimal image can be acquired also by reflecting previous imaging information or changing another control mechanism depending on a magnitude of the field angle.

Further, the resetting involves changing of another OCT control parameter. For example, the resetting also includes thinning-out during a scan for setting a size of the image appropriately. The above-mentioned drive control portion 180 configured to drive and control the coherence gate stage 121 forms an optical path length difference changing unit configured to change the optical path length difference between the optical path length of the measuring light and the optical path length of the reference light in the optical system. Further, the optical path length difference is one of the control parameters, which allows the above-mentioned switching unit to switch the optical path length difference when the insert lens 193 is inserted into the measuring optical path.

Further, an increase in the imaging time period causes an influence of an eye movement, and hence the resetting includes increasing of the number of layers to be superimposed. In other words, a display control parameter used when the tomographic image is displayed by a display control unit as described above is also included in at least one control parameter switched by the switching unit when the insert lens 193 is inserted into the measuring optical path.

Further, in regard to the imaging of the OCT image, there is known an SLO tracking technology for conducting tracking by using the fundus image obtained by the SLO portion 140, to thereby conduct registration at the time of generation of the B-scan image. The insertion of the insert lens 193 into the measuring optical path causes a change in the scanning speed of the measuring light on the fundus at the time of the OCT image acquiring. This requires the scanning speed of the SLO measuring light, a data acquisition timing, a data acquisition rate, and the like to be changed so as to correspond to the changed magnification of the field angle described above even in a case of using the SLO tracking technology. Also in this case, it is preferred that those control parameters be changed in the same manner as in the above-mentioned examples of resetting of the control condition.

### Changing of Processing Condition: Switching of Processing Parameter of Calculation Processing Portion

Next, the switching of the processing parameter of the calculation processing portion is described.

### (1) Signal Processing Parameter 1: Switching of Dispersion Compensation Parameter

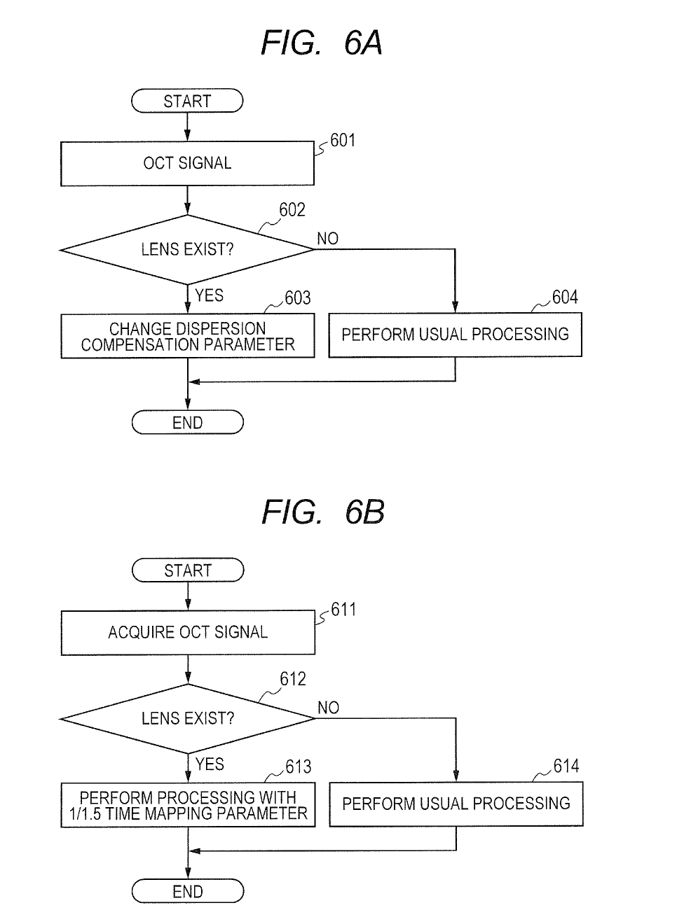

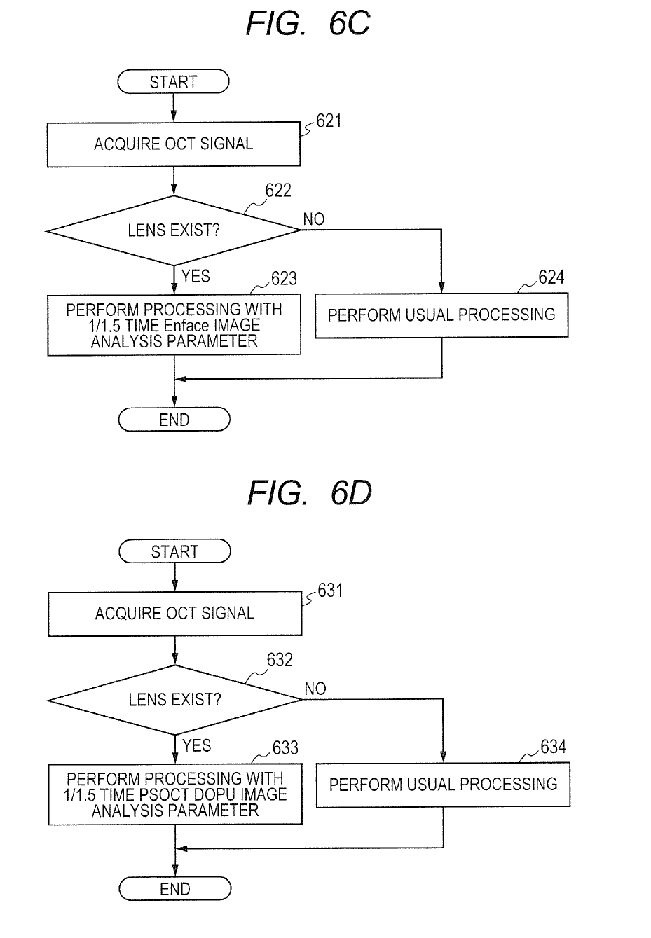

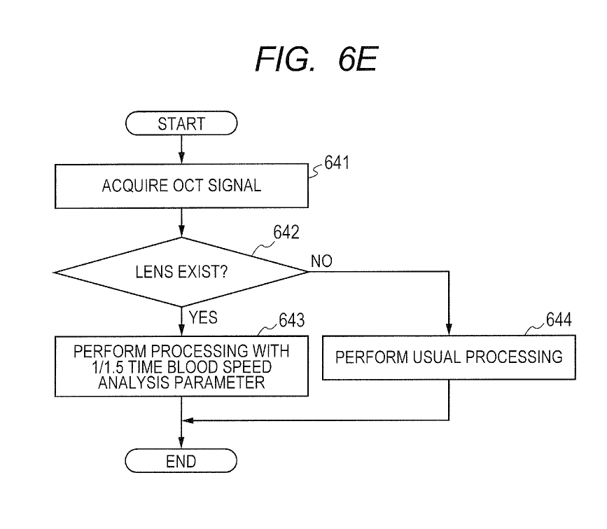

At the time of the imaging of the OCT image, the insertion of the insert lens 193 into the measuring optical path causes a difference between dispersion on the measuring light side and dispersion on a reference light side, which causes image deterioration. In order to prevent the image deterioration, it is preferred that the dispersion compensation parameter used at a time of the signal processing be reset and changed. A specific example of a process of such resetting of the dispersion compensation parameter is described below with reference to a flowchart illustrated in FIG. 6A.

The OCT signal output from the line camera 129 is obtained (Step 601), and the information on the presence or absence of the insert lens 193 on the measuring optical path is obtained based on the OCT signal (Step 602). When it is determined in Step 602 that the insert lens 193 is inserted in the measuring optical path, the flow advances to Step 603. In Step 603, a search is made for a site where a PSF exhibits a minimum half-value width, and a parameter used for dispersion compensation is reset. When it is determined in Step 602 that the insert lens 193 does not exist on the measuring optical path, the flow advances to Step 604, where the OCT image is constructed with a usual parameter.

Note that, in this embodiment, the resetting of the dispersion compensation parameter is handled by the signal processing. However, a manner of the dispersion compensation is not limited to this form, and the dispersion compensation can also be conducted with higher accuracy by, for example, inserting the same lens into a reference optical path side.

### (2) Signal Processing Parameter 2: Switching of Number of Sampling Interference Light

In a case of using a swept source OCT (SS-OCT) formed of a detector for differential detection with a wavelength sweeping light source used as a light source, the number of sampling of the interference light may be included as the signal processing parameter. In this case, it is preferred that the above-mentioned switching unit switch the number of sampling of the interference light so as to correspond to the changed field angle when the insert lens 193 is inserted into the measuring optical path. At this time, as described above, when the OCT imaging is conducted with a wide field angle, it is preferred that the depth-direction imaging range be set longer than the depth-direction imaging range of the OCT image of the usual field angle so that the curved fundus falls within the imaging range as much as possible (see FIG. 2B′). Therefore, in order to change the field angle so that the field angle becomes wider, it is preferred to increase the number of sampling of the interference light. This allows the tomographic image to be obtained, for example, within the depth-direction imaging range longer than the depth-direction imaging range of the OCT image of the usual field angle, and hence the curved fundus easily falls within the imaging range. Note that, the number of sampling referred to herein represents a frequency of a so-called k-clock, and the increasing of the number of sampling corresponds to increasing of the frequency of the k-clock.

### (3) Signal Processing Parameter 3: Switching of Gain of Output Signal from Line Camera

Further, in a case of using the SD-OCT for detecting a light source having a spectrum width through use of a spectroscope, a gain obtained when the output signal from the line camera 129 is processed may be included as the signal processing parameter. In this case, it is preferred that the switching unit switch the gain of the output signal so as to correspond to the change of the field angle. At this time, when the OCT imaging is conducted with a wide field angle, for example, a vitreous body existing on the retina is often wished to be observed. Therefore, in order to change the field angle so that the field angle becomes wider, it is preferred to increase the gain. This allows the tomographic image to be obtained, for example, with a higher contrast than the OCT image of the usual field angle, which allows the tomographic image to be obtained with an emphasis put on the vitreous body.

### (4) Analysis Processing Parameter 1: Switching of Size of Two-Dimensional Image Such as Map

Further, as illustrated in FIG. 2A, FIG. 2A′, FIG. 2A″, FIG. 2B, FIG. 2B′, and FIG. 2B″, the insertion of the insert lens 193 allows the wide-field-angle OCT image illustrated in FIG. 2B′ to be acquired. The wide-field-angle OCT image has a lower resolution power (in the x direction and also in the y direction in FIG. 2A, FIG. 2A′, FIG. 2A″, FIG. 2B, FIG. 2B′, and FIG. 2B″) than the OCT image of the usual field angle illustrated in FIG. 2A′. This is because the sampling period is the same irrespective of the increased field angle (imaging distance), and hence a distance per unit pixel is different. In this embodiment, in consideration of such a phenomenon, an example of assisting an appropriate diagnosis by changing an analysis numerical value obtained from the OCT image is described.

In recent years, the OCT image of the subject is acquired and compared with a normative database (database regarding a normal eye; hereinafter referred to as “NDB”), to thereby inspect presence or absence of a disease of the subject. For example, to diagnose a glaucoma, a physician compares a thickness map of a nerve fiber layer obtained from the OCT signal with the NDB. Therefore, in order to form the thickness map of the nerve fiber layer, it is preferred to appropriately set distances exhibited when the OCT image is displayed in the x direction and in the y direction. A process of appropriately setting a display distance for such an NDB analysis is described with reference to a flowchart illustrated in FIG. 6B.

The OCT signal output from the line camera 129 is obtained (Step 611), and the information on the presence or absence of the insert lens 193 on the measuring optical path is obtained based on the OCT signal (Step 612). When it is determined in Step 612 that the insert lens 193 is inserted in the measuring optical path, the flow advances to Step 613. At this time, processing for setting the distances for the map in the x direction and the y direction to become 1/1.5 times (because the field angle becomes 1.5 times larger) (processing for decreasing a size thereof) is executed. When it is determined in Step 612 that the insert lens 193 does not exist on the measuring optical path, the flow advances to Step 614, where the OCT image is constructed under a usual analysis condition.

Further, an Enface (C-scan) image analysis causes the same phenomenon as the analysis using the map. Therefore, it is preferred that the same processing be executed to construct the OCT image. A specific example of such analysis processing is described with reference to a flowchart illustrated in FIG. 6C.

The OCT signal output from the line camera 129 is obtained (Step 621), and the information on the presence or absence of the insert lens 193 on the measuring optical path is obtained based on the OCT signal (Step 622). When it is determined in Step 622 that the insert lens 193 is inserted in the measuring optical path, the flow advances to Step 624. At this time, processing for setting the distances for the Enface image in the x direction and the y direction, which are used as the analysis parameter for an Enface image, to become 1/1.5 times (because the field angle becomes 1.5 times larger) (processing for decreasing a size thereof) is executed. When it is determined in Step 622 that the insert lens 193 does not exist on the measuring optical path, the flow advances to Step 624, where the OCT image is constructed under a usual analysis condition.

### (5) Analysis Processing Parameter 2: Others

It is preferred that processing for appropriate setting conducted at a time of each analysis described above be also executed at a time of phase correction processing, at a time of degree of polarization uniformity (DOPU) processing conducted by a polarization OCT apparatus, at a time of blood speed processing conducted by a Doppler OCT apparatus, or the like. Note that, a DOPU is a parameter indicating uniformity of polarization, and is obtained for each ROI. A process of the processing for appropriate setting conducted at a time of the DOPU processing and at a time of the blood speed processing is illustrated in flowcharts of FIG. 6D and FIG. 6E. In the processing for appropriate setting, the OCT signal output from the line camera 129 is obtained (Step 631 and Step 641), and the information on the presence or absence of the insert lens 193 on the measuring optical path is obtained based on the OCT signal (Step 632 and Step 642). When it is determined in Step 632 or Step 642 that the insert lens 193 is inserted in the measuring optical path, the flow advances to Step 633 or Step 643. At this time, processing for setting, for example, a length of a side of a ROI, which is used as the analysis parameter for a DOPU image, to become 1.5 times (because the field angle becomes 1.5 times larger) is executed, or processing for setting a blood speed obtained through use of the OCT image having a wide field angle, which is used as the analysis parameter for the blood speed, to become 1/1.5 times (because the field angle becomes 1.5 times larger) is executed. When it is determined in Step 632 or Step 642 that the insert lens 193 does not exist on the measuring optical path, the flow advances to Step 634 or Step 644, where the OCT image is constructed under a usual analysis condition. Note that, in this case, it is assumed that the pixel number of the tomographic image displayed on a monitor is fixed even when the field angle is changed. In such a case, the above-mentioned processing is not required as long as processing for setting a length of one pixel to become 1.5 times (because the field angle becomes 1.5 times larger) is executed in advance.

As described above, an appropriate analysis numerical value is allowed to be obtained by causing each of those parameters used for the processing to correspond to the presence or absence of the insert lens 193 on the measuring optical path. Further, it should be understood that adaptation to the above-mentioned phenomena is allowed also at a time of setting a threshold value for segmentation, another function OCT, or another analysis condition. For example, threshold values of the contrast, a luminance, and the like, which are used to distinguish a boundary between a plurality of layers included in the tomographic image when the tomographic image is subjected to the analysis processing, are each included as one of the analysis processing parameters as well. In this case, it is preferred that the above-mentioned switching unit switch the threshold value between both end portions and a central portion within the tomographic image when the insert lens 193 is inserted into the measuring optical path. Further, at this time, it is preferred that those threshold values for the switching be stored in a table corresponding to the power or the like of the insert lens 193 in advance.

Further, the signal processing portion 190 may be provided with a module area that functions as a value determination unit configured to determine a value of at least one parameter based on an insertion position of the insert lens 193 inserted in the measuring optical path. In this case, the operator's input, use of a dedicated detector, or the like is conceivable for the detection of the insertion position. Further, the switching unit used in this case may switch the at least one parameter to the determined value when the insert lens 193 is inserted into the measuring optical path. Therefore, the tomographic image suitable for the insertion position is expected to be obtained.

### Changing of GUI Display Condition: Switching of Display Control Parameter

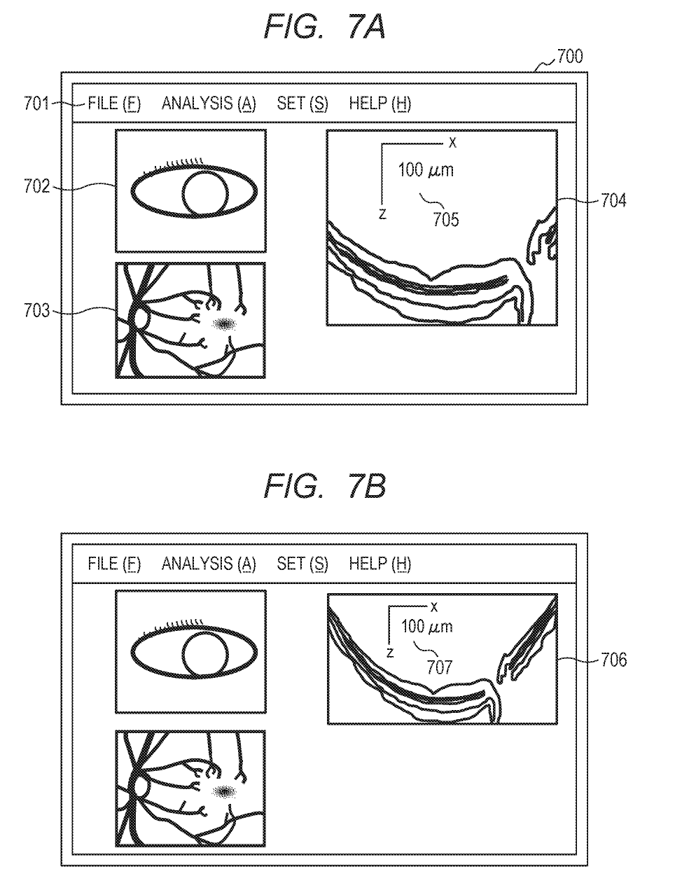

In this embodiment, the insertion of the insert lens 193 into the measuring optical path causes the scanning ranges of the SLO image and the OCT image in the x direction and the y direction to become 1.5 times. This requires a scale bar of the image to be changed when the GUI display is conducted. The changing of the scale bar is described with reference to FIG. 7A and FIG. 7B.

First, an example of the usual GUI display is illustrated in FIG. 7A. On a GUI screen 700, a GUI header 701 includes “file”, “analysis”, “set”, and “help”, and an anterior ocular segment monitor image 702, an SLO image 703, and an OCT image 704 are also displayed. Further, a scale bar 705 is displayed together on the OCT image (B-scan image) 704. When the OCT imaging is conducted with a wide field angle, such an image as illustrated in FIG. 7B as an OCT image 706 is allowed to be obtained. In this case, the scale bar is required to be changed to a scale bar 707 for the OCT image.

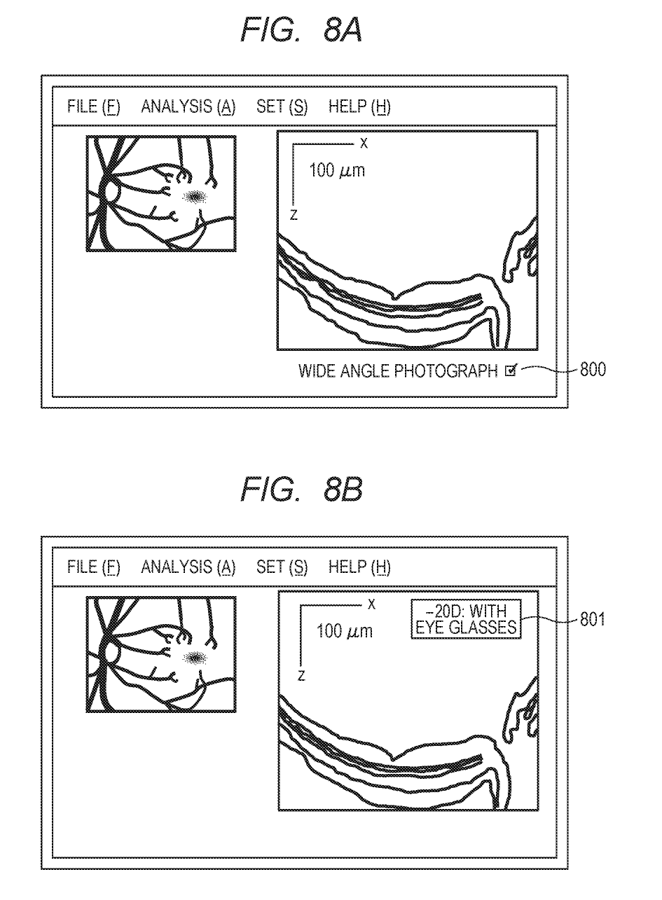

Further, a fact that the field angle has become wider is required to be displayed together, in regard to which, such display manners as exemplified in FIG. 8A and FIG. 8B are also effective. For example, the appropriate diagnosis of the physician is assisted also by checking by putting a check mark on a button 800 for a wide angle image as illustrated in FIG. 8A, or displaying the display “−20 D: with eyeglasses” indicating that the wide angle image has been obtained on the image as illustrated in FIG. 8B.

Further, when the insert lens 193 is inserted in the measuring optical path, the field angle becomes wider, and hence it is preferred to change γ, the contrast, or the like as an image display parameter. Further, this holds true of the display of a map indication, a 3D image, the Enface image, or the like, and the same effects are produced by providing support using the above-mentioned display. Further, it is preferred that the scale bar (scale indication), the fact of being the image acquired with a wide field angle, an association between the image and the information, a degree (1.5 times) of the wide field angle, or the like be displayed in the same manner.

Note that, the embodiment is described above by taking an exemplary case where the SD-OCT for detecting the light source having a spectrum width through use of the spectroscope is used for the OCT portion 100 used for the ophthalmic apparatus. However, the same effects are produced even in the case of using the SS-OCT formed of the detector for differential detection with the wavelength sweeping light source used as the light source.

### Recommended Mode: OCT Focus

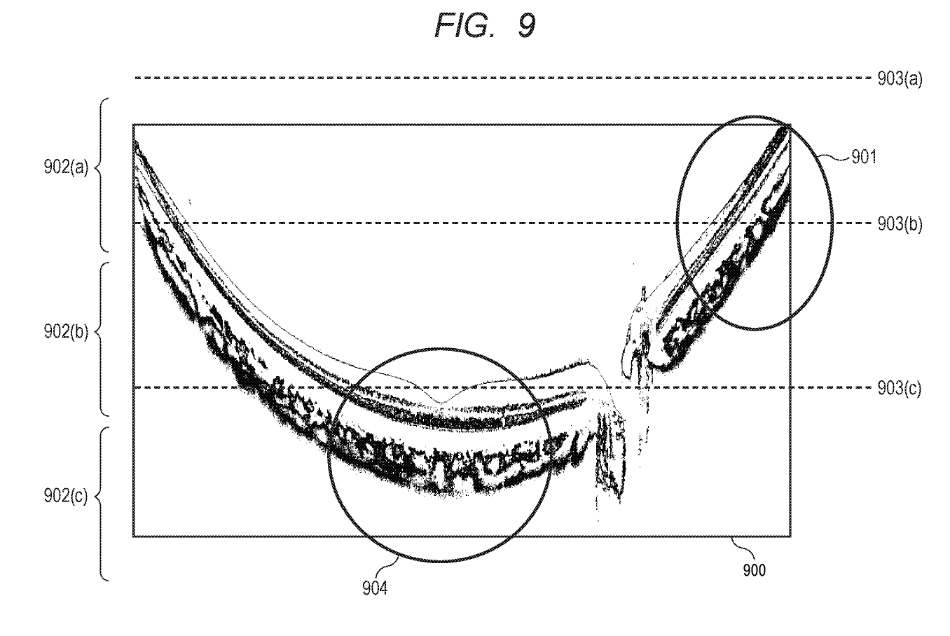

As described above, when the power of the insert lens 193 is set to approximately −30 D, the field angle of the OCT image becomes wider as exemplified in FIG. 9. When the field angle becomes wider, the depth information is required to be increased so as to correspond to a fundus arch portion 901 of the eyeball within the obtained image. However, when the processing for increasing the depth information illustrated in FIG. 5B is conducted, a limitation is imposed on an appropriate focus area of the measuring light at a time of the acquisition of the OCT signal. Therefore, a position out of focus exists within a measurement region, which causes a luminance difference within the image.

Now, processing for obtaining an image exhibiting no luminance difference is described. In this processing, a region 902(a) in the depth direction is first brought into focus, and the OCT image is acquired in this region. Subsequently, a region 902(b) and a region 902(c) are brought into focus in order, and the OCT images are acquired in the respective regions. After that, the OCT images acquired in the respective layers are superimposed on each other, which allows the acquisition of the appropriate OCT image having sufficient depth information with a wider field angle.

In this case, it is preferred to determine the number of layers of a plurality of tomographic images to be superimposed on each other at each of a plurality of imaging positions so as to reduce a difference in luminance among the plurality of imaging positions in the depth direction of the tomographic image based on an optical characteristic of the insert lens 193. Further, it is preferred that the number of layers to be superimposed be determined by a module area defined as a number-of-layers determination unit constructed to execute this function in the signal processing portion 190. This allows provision of the OCT image that has the sufficient depth information with a wide field angle and exhibits no sense of incompatibility at a joint portion.

Further, a higher quality OCT image is obtained by further matching the above-mentioned control with the control of the C-Gate. Specifically, the region 902(a) is brought into focus, and the C-Gate position is set as a position 903(a), to acquire a plurality of OCT images. Subsequently, the region 902(b) is brought into focus, and the C-Gate position is set as a position 903(b), to acquire a plurality of OCT images. Then, the region 902 (c) is brought into focus, and the C-Gate position is set as a position 903(c), to acquire a plurality of OCT images. Three kinds of superimposed OCT images obtained by the above-mentioned operation are used to be further reconstructed into one OCT image, which allows the acquisition of the OCT image that is deep and has an optimally wide angle.

Note that, the control described above may be conducted from the position of the vitreous body within the eye to be inspected.

### Recommended Mode: Setting Film Thickness Appropriately

As in the OCT image illustrated in FIG. 9, when an OCT image 900 is acquired with a wide field angle, an OCT image central portion 904 and the fundus arch portion 901 differ from each other in the imaging condition. An accurate measurement of a film thickness at an end portion becomes difficult due to an optical distortion, an optical distance based on an incident angle, or an interference signal based on a primary scattered light. Therefore, when it is detected that the insert lens 193 has been inserted into the measuring optical path, the following processing enables appropriate assistance of the diagnosis.

In other words, power information on the insert lens 193 is first obtained by the user's input or by a lens sensing function. Subsequently, each optical performance within an optical scanning area is calculated from cornea data on the subject. After that, dependence is put on the field angle from the central portion, and the above-mentioned optical parameter is reflected in the calculation of the film thickness of each layer of the retina. This recommended mode is reflected in the flow of each series of processing described above, to allow the film thickness of each layer to be obtained accurately without dependence on a location of the retina. Note that, the above-mentioned operation is executed by a module area within the signal processing portion 190, which functions as a correction unit configured to correct a distortion of the tomographic image, based on the optical characteristic of the insert lens 193 and the optical characteristic of a cornea of the eye to be inspected.

As described above, in a case where the acquisition of the OCT image with a wide field angle is allowed when the subject inserts the insert lens 193 into the measuring optical path with the eyeglasses, appropriate control and processing are conducted to thereby allow the acquisition of the OCT image having a high resolution power with a wide field angle. Note that, the above description of the embodiment is directed to the case where the insert lens 193 is −20 D, but this value is not limited thereto, and may be +20 D. In that case, the field angle becomes small, and hence the parameter may be set in a manner opposite to the above description.

### Other Embodiments

Note that, the present invention is not limited to the above-mentioned embodiment, and may be conducted with various changes and modifications within the scope that does not depart from the gist of the present invention. For example, the description of the above-mentioned embodiment is directed to the case where an object to be inspected is an eye, but the present invention may be applied to an object to be inspected such as a skin or an organ other than the eye. In this case, the present invention has a mode as medical equipment such as an endoscope other than the ophthalmic apparatus. Accordingly, it is desired that the present invention be grasped as a tomographic imaging apparatus exemplified by the ophthalmic apparatus, and the eye to be inspected be grasped as one mode of the object to be inspected.

Further, another embodiment of the present invention may be configured as an optical tomographic imaging system including: an optical tomographic imaging apparatus; and an optical member for changing a field angle to be attached by the subject in order to change the field angle of the image acquiring area of the tomographic image of the eye to be inspected. At this time, examples of the optical member for changing a field angle to be attached by the subject include the eyeglasses and the contact lens. This allows the field angle of the image acquiring area of the tomographic image to be changed with ease even in the optical tomographic imaging apparatus or the like designed without assumption of the attachment of the insert lens or the adapter lens. Note that, at an ophthalmic medical site, in general, the eye to be inspected is imaged after the subject is asked to take off the eyeglasses or the contact lens in order to prevent the ghost or the like due to the reflection of the lens.

In this case, an optical tomographic imaging system according to the above-mentioned another embodiment may be grasped as including: an optical tomographic imaging apparatus including a light source, an optical splitter configured to split a light emitted from the light source into a measuring light and a reference light, a scanning unit configured to scan an eye to be inspected with the measuring light, an optical system configured to irradiate the eye to be inspected with the measuring light through the scanning unit, a detector configured to receive an interference light between a return light of the measuring light from the eye to be inspected and the reference light, and a calculation processing portion configured to process an output signal from the detector, to acquire a tomographic image of the eye to be inspected; and an optical member for changing a field angle to be attached by a subject in order to change the field angle of an image acquiring area of the tomographic image.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-003421, filed Jan. 9, 2015, which is hereby incorporated by reference herein in its entirety.

### REFERENCE SIGNS LIST

100: OCT portion, 129: line camera, 140: SLO portion, 160: anterior ocular segment observation portion, 170: internal fixation lamp portion, 180: drive control portion, 190: signal processing portion, 191: display control portion, 192: display portion, 193: insert lens, 194: switching portion, 200: control portion

1. An optical tomographic imaging apparatus, comprising:
a light source;
an optical splitter configured to split a light emitted from the light source into a measuring light and a reference light;
a scanning unit configured to scan an object to be inspected with the measuring light;
an optical system configured to irradiate the object to be inspected with the measuring light through the scanning unit;
a detector configured to receive an interference light between a return light of the measuring light from the object to be inspected and the reference light;
a calculation processing portion configured to process an output signal from the detector, to thereby acquire a tomographic image of the object to be inspected;
a determination unit configured to determine whether or not an optical member for changing a field angle is inserted between the scanning unit and the object to be inspected in order to change the field angle of an acquiring area of the tomographic image; and
a switching unit configured to switch a value of at least one parameter among a control parameter of a control portion configured to control the optical tomographic imaging apparatus, a signal processing parameter of the calculation processing portion, an image processing parameter, and an analysis processing parameter, based on a determination result from the determination unit.

2. An optical tomographic imaging apparatus according to claim 1, wherein:
the control parameter comprises a scanning speed of the measuring light exhibited by the scanning unit; and
the switching unit is further configured to switch the scanning speed when the optical member for changing a field angle is inserted.

3. An optical tomographic imaging apparatus according to claim 1, wherein:
the optical system comprises an optical path length difference changing unit configured to change an optical path length difference between an optical path length of the measuring light and an optical path length of the reference light;
the control parameter comprises the optical path length difference; and
the switching unit is further configured to switch the optical path length difference when the optical member for changing a field angle is inserted.

4. An optical tomographic imaging apparatus according to claim 1, further comprising a display control unit configured to display the tomographic image on a display unit,
wherein a display control parameter used when the tomographic image is displayed by the display control unit is included in at least one control parameter switched by the switching unit when the optical member for changing a field angle is inserted.

5. An optical tomographic imaging apparatus according to claim 1, wherein:
the signal processing parameter comprises a number of sampling of the interference light; and
the switching unit is further configured to switch the number of sampling of the interference light when the optical member for changing a field angle is inserted.

6. An optical tomographic imaging apparatus according to claim 1, wherein:
the signal processing parameter comprises a gain obtained when the output signal from the detector is processed; and
the switching unit is further configured to switch the gain of the output signal when the optical member for changing a field angle is inserted.

7. An optical tomographic imaging apparatus according to claim 1, wherein:
the analysis processing parameter comprises a threshold value used to distinguish a boundary between a plurality of layers included in the tomographic image when the tomographic image is subjected to analysis processing; and
the switching unit is further configured to switch the threshold value between both end portions and a central portion within the tomographic image when the optical member for changing a field angle is inserted.

8. An optical tomographic imaging apparatus according to claim 1, further comprising an input unit configured to make an input of a fact that the optical member for changing a field angle is inserted by an operator,
wherein the determination unit is further configured to determine that the optical member for changing a field angle is inserted based on the input made by the input unit.

9. An optical tomographic imaging apparatus according to claim 1, wherein the determination unit is further configured to determine whether or not the optical member for changing a field angle is inserted based on the output signal from the detector.

10. An optical tomographic imaging apparatus according to claim 1, further comprising a value determination unit configured to determine the value of the at least one parameter based on an insertion position of the optical member for changing a field angle inserted in an optical path of the measuring light,
wherein the switching unit is further configured to switch the at least one parameter to the determined value when the optical member for changing a field angle is inserted.

11. An optical tomographic imaging apparatus according to claim 1, further comprising a number-of-layers determination unit configured to determine a number of layers of a plurality of tomographic images to be superimposed on each other in each of a plurality of imaging positions so as to reduce a difference in luminance among the plurality of imaging positions in a depth direction of the tomographic image based on an optical characteristic of the optical member for changing a field angle.

12. An optical tomographic imaging apparatus according to claim 1, wherein:
the object to be inspected comprises an eye to be inspected; and
the optical tomographic imaging apparatus further comprises a correction unit configured to correct a distortion of the tomographic image based on an optical characteristic of the optical member for changing a field angle and an optical characteristic of a cornea of the eye to be inspected.

13. An optical tomographic imaging apparatus according to claim 1, wherein:
the object to be inspected comprises an eye to be inspected;
the optical tomographic imaging apparatus further comprises a second detection portion configured to receive a return light from the eye to be inspected in order to acquire at least one of an anterior ocular segment image of the eye to be inspected or a fundus image of the eye to be inspected; and
the determination unit is further configured to determine whether or not the optical member for changing a field angle is inserted based on an output signal from the second detection portion.

14. An optical tomographic imaging apparatus according to claim 1, wherein:
the object to be inspected comprises an eye to be inspected; and
the scanning unit is arranged at a position conjugate with an anterior ocular segment of the eye to be inspected, and is further configured to scan a fundus of the eye to be inspected with the measuring light.

15. An optical tomographic imaging apparatus according to claim 1, wherein the optical member for changing a field angle comprises any one of eyeglasses, a contact lens, and an adapter lens mounted on the optical tomographic imaging apparatus.

16. A non-transitory tangible medium having recorded thereon a program for causing a computer to be executed as the switching unit included in the optical tomographic imaging apparatus of claim 1.

17. A method of controlling an optical tomographic imaging apparatus,
the optical tomographic imaging apparatus comprising:
a light source;
an optical splitter configured to split a light emitted from the light source into a measuring light and a reference light;
a scanning unit configured to scan an object to be inspected with the measuring light;
an optical system configured to irradiate the object to be inspected with the measuring light through the scanning unit;
a detector configured to receive an interference light between a return light of the measuring light from the object to be inspected and the reference light; and
a calculation processing portion configured to process an output signal from the detector, to thereby acquire a tomographic image of the object to be inspected,
the method comprising:
determining whether or not an optical member for changing a field angle is inserted between the scanning unit and the object to be inspected in order to change the field angle of an acquiring area of the tomographic image; and
switching a value of at least one parameter among a control parameter of a control portion configured to control the optical tomographic imaging apparatus, a signal processing parameter of the calculation processing portion, an image processing parameter, and an analysis processing parameter, based on a determination result in the determining.

18. A non-transitory tangible medium having recorded thereon a program for causing a computer to execute the control method for an optical tomographic imaging apparatus of claim 17.

19. An optical tomographic imaging system, comprising:
an optical tomographic imaging apparatus comprising:
a light source;
an optical splitter configured to split a light emitted from the light source into a measuring light and a reference light;
a scanning unit configured to scan an eye to be inspected with the measuring light;
an optical system configured to irradiate the eye to be inspected with the measuring light through the scanning unit;
a detector configured to receive an interference light between a return light of the measuring light from the eye to be inspected and the reference light; and
a calculation processing portion configured to process an output signal from the detector, to thereby acquire a tomographic image of the eye to be inspected; and
an optical member for changing a field angle to be attached by a subject in order to change the field angle of an acquiring area of the tomographic image.
